(19) United States
(12) Patent Application Publication
(10) Pub. No.: US 2021/0267521 A1
(43) Pub. Date: Sep. 2, 2021

(54) INDIVIDUALIZED METHOD, SYSTEM, APPARATUS AND PROBE FOR ELECTROENCEPHALOGRAPHY

(71) Applicant: Mindseed IVS, Greve (DK)

(72) Inventor: Christopher James Luscher, Valby (DK)

(21) Appl. No.: 17/254,541

(22) PCT Filed: Jun. 25, 2019
(86) PCT No.: PCT/DK2019/050204

§ 371 Date: Dec. 21, 2020

(30) Foreign Application Priority Data
Jun. 25, 2019 (DK) PA201870436

(51) Int. Cl. A61B 5/291; A61B 5/00; A61B 5/369; A61B 5/257

(52) U.S. Cl. CPC A61B 5/291; A61B 5/7246; A61B 5/257; A61B 5/7225; A61B 5/6815; A61B 5/369

(57) ABSTRACT

A region on the head is examined with respect to the different muscles present in the region, and correlation areas are defined, where not two correlation areas relate to the same muscle in the region. The EEG probes are then produced individually for the examined human in order to optimize positioning of the electrodes on the different correlation areas. This way, signals from the muscles can be filtered out relatively easy when combining the signals from the different electrodes because the signals are not correlated between the electrodes, in contrast to the EEG signals.

### CROSS REFERENCE TO RELATED APPLICATIONS

This application is the U.S. national stage of PCT/DK2019/050204 filed Jun. 25, 2019, which claims priority of Denmark patent application PA201870436 filed Jun. 25, 2018, both of which are hereby incorporated by reference in their entirety.

### FIELD OF THE INVENTION

The present invention relates to an individualized method, system and apparatus for electroencephalography, EEG.

### BACKGROUND OF THE INVENTION

Biomarkers are quantifiable data collected from the body to account for a given physiological response. Examples of methods, where electrodes are used for collecting such responses, are electrocardiography (ECG), electroencephalography (EEG), or skin resistivity, also called galvanic response of the skin. These data are useful for understanding and monitoring the body and a given physiological response.

For EEG, different types of apparatuses exist. Some are of the type with a large number of electrodes, which are positions around the head, typically 10 to 30 electrodes but in some cases up to 256 electrodes. Recently, simpler systems have been marketed, especially in order to make the systems user friendly and suitable for daily use.

In some cases, the development has been improved with respect to improved design and ease of use. An example is the EEG with the trade name Insight™ from the com pang Emotiv®, see https://www.emotiv.com/insight/, which relates to a 6 channel EEG as shown in FIG. 1a, which is a reproduction of the drawing from the Internet page https://emotiv.zendesk.com/hc/en-us/articles/204942089-How-come-my-headset-doesn-t-turn-on-. The Insight™ has the overall appearance of a headphone and comprises electrodes at a base as well as at the end of four arms that extend from the base.

In other cases, the development has taken a direction of minimization. An example of a smaller EEG electrode arrangement is marketed as cEEGrid™, and described in detail on the Internet site http://ceegrid.com. The system was published in 2015 by Debener et al. in the article “Unobtrusive ambulatory EEG using electrodes around the ear” published in Scientific Reports|5: 16743|DOI: 10.1038/srep16743, currently available on the internet site http://ceegrid.com/download/Debener15.pdf. FIG. 1b is a reproduction of the drawing from the Internet page http://ceegrid.com/home/concept/

An even smaller EEG system is available for use inside the ear, such as disclosed on the Internet site http://ear-eeg.org/. A copy of the system from this particular Internet site is reproduced on FIG. 1c.

A further small alternative is found on the Internet site https://www.uneeg.com. It discloses a two channel EEG with an implanted electrode and an external receiver for attachment to the head. The system is explained in detail in the Internet site https://www.uneeg.com/en/products/24-7eeg/overview. FIG. 1d is a reproduction from this site.

These systems have advantageous with respect to design and size as compared to previous more complex scientific and diagnostic systems. Although, they fit various head sizes and forms, they are not optimized with respect to such parameters, especially size. For example, these systems are not equally useful for small heads of children and large adult heads. Accordingly, there is room for improvement of the systems with respect to individual adjustment as well as optimization of EEG signals.

### SUMMARY OF THE INVENTION

It is an objective of the invention to provide an improvement in the art. In particular, it is an objective to provide an improved electrode system, especially for EEG. It is a further objective to provide a method for optimizing the signal collection for EEG. These and further objectives are achieved with an apparatus, a system and methods as described in more detail in the following.

The apparatus, system and methods are based on individually sized probes for measuring EEG, and potentially also ECG. A further option is use of the system for measuring galvanic response from the skin.

The customization by individually sizing the probes takes offset in the actual size and shape of the head of the individual human, as there are substantial variations of the head size and shape among a plurality of human individuals. Especially, the sizes between children, women and men vary substantially, and children's heads grow remarkably within short time, which makes adjustments necessary in order to obtain optimum signals when measuring EEG signals despite growth of the child.

Although, prior art EEG probes can be used on various sizes of heads, for example because they are mounted on flexible frames, thorough study has revealed that the prior art probes suffer from reduced precision due to interfering signals from the muscles under the electrodes. Especially in the case where two electrodes are placed above or near the same muscle or muscle group, the electrical signals from the muscle create unwanted correlated overlay on the EEG signals as well as electrode saturation, and there is a risk for artefacts and distortion in the signals, leading to misinterpretation of the EEG signals. It is therefore a further objective of the invention to at least reduce if not avoid such distortion.

For these reasons, there has been taken into account the specific locations and potentially also size and direction of the head muscles for each specific human individual in the region on the head where the measurements are performed. The electrodes are then placed on different muscles such that correlation of electrical raw signals in the electrodes from a muscle is avoided.

Herein, the term “raw signal” is used for the signal that is received from the electrodes, and the term“EEG signal” is used for a signal resembling brain activity and obtained after extraction from the raw signal by using filtering techniques, including correlation criteria between the raw signals from the multiple electrodes.

In practice, a region on the head is examined with respect to the different muscles present in the region, and correlation areas are defined, where not two correlation areas relate to the same muscle in the region. Raw signals from electrodes on different correlation areas are then expected not to contain signals from the same muscle, such that the muscle signals can be filtered out relatively easy when combining the signals from the different electrodes because the muscle signals are not correlated, or at least there is only very little correlation. In contrast thereto, EEG signals from the various electrodes are strongly correlated.

Although, the invention also applies to other head regions, a region of special interest is around the ear, and a thorough examination of the head region around the ear reveals individual information related to locations of the corresponding muscles, in particular the anterior auricular muscle, the posterior auricular muscle, the Temporoparietalis, and the superior auricular muscle.

Further as part of the invention, corresponding correlation areas are defined:

an anterior correlation area A on the anterior auricular muscle, for example where the pinna, soft-tissue of the ear, adheres to the anterior auricular muscle;
a posterior correlation area B on the posterior auricular muscle, for example where the posterior auricular muscle attaches to the temporal bone behind the external auditory meatus and below the zygomatic process and above the mastoid process;
a Temporoparietalis correlation area C on the Temporoparietalis, for example located where the pinna adheres on top of the Temporoparietalis muscle;
a superior correlation area D on the superior auricular muscle, for example where the pinna adheres on top of the superior auricular muscle.

For the EEG probe with a plurality of electrodes, an electrode is provided on each of at least two correlation areas, for example three or four correlation areas. As the correlation areas relate to different muscles, unwanted interfering signals from the muscles can be relatively easily be filtered out, as already explained above.

In practice, the EEG probe with the electrodes is placed, for example glued, against the skin of the head at the location of the different correlation areas for the at least two selected muscles, for example three or four selected muscles, and the electrical signals in the head are measured by the electrodes. From the measured electrical raw signals, the EEG signals are extracted by correlating the signals from the at least two electrodes, typically after digitization of the raw signals. Further, the electrical influence from the at least two selected muscles is reduced or even removed by selectively ng out those parts of the signals that are uncorrelated between the electrodes.

In some embodiments, a probe is provided on only one side of the head, in particular in a region around the ear.

In order to optimize the function of the probes on an individual basis and achieve proper fitting the head of each individual, the EEG probes are provided different in size and potentially also different in shape based on the data from the examination.

For example, the EEG probe is provided as an individually sized flexible support sheet with electrodes, optionally printed by metallic ink, on the support sheet at the locations for the different correlation areas related to the at least two selected muscles.

Typical materials for the sheet are polymers, for example foams or rubbers. A useful material is silicone. Optionally, the sheet is provided as a polymer film having glue on one side,

Optionally, more than a single electrode is provided on a correlation area. The electrodes are grouped with one group on each of the selected correlation areas. Optionally, more than one correlation area comprises group a plurality of electrodes, for example two or three electrodes on each of the at least two selected correlation areas.

In some embodiments, the method comprises attaching the EEG probe with a first and a second electrode on a first and a second of the correlation areas, respectively, and at least one further electrode on the first of the correlation areas.

For example, differential signals are calculated between the first electrode and the at least one further electrode on the first of the correlation areas, and from the differential signal, a direction towards the source of the extracted EEG signal inside the brain of the human individual is determined.

When three electrodes are used on the same correlation area, a useful method for fmd-ing a direction towards the EEG signal source is triangulation, where amplitudes of the EEG signals from the three electrodes are compared, and the highest and lowest amplitudes, respectively, are regarded as belonging to the electrodes closest and farthest from the source point.

In some embodiments, the method comprises supplementing the EEG signals by additionally extracting ElectroCardioGraphy, ECG, signals from the electrical signals measured by the EEG probes. Other signals of interest are galvanic skin responses, which can also be measured by the electrodes when measuring current through the skin from one electrode to the other.

In practical examples for production of the EEG probes, as a basis, data are received for each specific human individually, where the data contain individual information related to locations of at least two head muscles and the corresponding correlation areas. On the basis of the received data, an individually sized EEG probe is produced for each human individuals, where each EEG probe comprises electrodes at locations on the EEG probe corresponding to the individual locations of the different correlation areas for the at least two muscles according to the specific individual information for each of the human individuals.

In some practical embodiments, a system is provided for measuring these EEG signals by individually sized EEG probes for human individuals, where the system comprises a digital database containing data with individual information related to locations of at least two head muscles for each specific of the human individuals. Further, the system comprises an individually sized EEG probe for each individual, the EEG probe comprising electrodes at locations on the EEG probe corresponding to the locations of the different correlation areas.

Advantageously, a computing device is provided for each individual, the computing device being functionally connected to the electrodes of one of the individually sized EEG probes for receiving the raw signals from the EEG probe.

The term computing device covers a device comprising

a data processing device for processing digital data on the basis of the raw signals from the electrode,
a data storage device for storage of processed digital data, and
optionally a display for illustrating processed data to a user.

The computing device is not necessary a single unit but can be provided as multiple units. In the following, an example is given in which part of the computing device is provided as a signal processing unit attached to the probe on the head of the user, and a further data processing unit remote from the probe, for example connected to the probe by a cable or by a wireless connection.

In order to provide digitized electrode signals, the raw signals from the electrodes are digitized by an analog-to-digital (A/D) converter. Optionally, a miniature A/D converter is integrated in the electrodes or provided on the electrodes. Alternatively, an A/D converter, for example a multichannel A/D converter, is provided as a module that is fixed to the probe and which receives the raw signal data from the various electrodes, typically through cables. The A/D conversion is typically made before filtering and correlating the signals from the various electrodes.

For example, the A/D converter is part of a signal processing unit that is attached to the probe. Such signal processing unit receives the raw signals, converts the raw signals to digital signals and performs a first signal processing, potentially correlating and filtering the signals. The digital signals after the first signal processing are then transmitted to the data processing unit for further data processing. For example, the processed data are displayed on a display with a user interface in order to ease interpretation of the data by the user.

As already outlined above, the computing device is configured for analyzing the received raw signals and extracting the EEG signal by correlating the raw signals from the at least two electrodes on the different correlation areas. Advantageously, the computing device is reducing the electrical influence from the at least two selected muscles by selectively filtering out those parts of the raw signals that are uncorrelated between the electrodes.

The invention also concerns an individualized apparatus for the method and system as described above. The apparatus is configured for measuring EEG signals by an individually sized EEG probe for a human individual. It comprises an EEG probe, as described above, which is individually sized for the human individual, based on data containing individual information related to locations of the head muscles.

A computing device is functionally connected to the electrodes of the EEG probe for receiving the signals from the EEG probe for analyzing the received signals and extracting the EEG signal, as described above. The computing device, or part of it, is advantageously portable by the human individual.

For example, the computing device comprises a smartphone as a receiver and processor of digital signal data, and the data processing function on the smartphone is provided as an installed computer application, typically called APP.

In order for the smartphone to receive the signals digitally, and preferably wirelessly, the signal processing unit comprises a transmitter, for example Bluetooth transmitter, and, as already outlined above, optionally also comprises a analog to digital converter for converting the analog raw signals from analog form to digital form prior to the converted signals being wirelessly sent to the smartphone.

For example, the computing device is provided as a combination of a smartphone and a dedicated signal processing unit, wherein the latter is used for raw signal collection from the EEG probe and the smartphone with a corresponding APP in communication with the signal processing unit is used for performing the calculation and analysis. In this case, the data collection can be made efficiently by a specially dedicated small, low-cost unit, potentially attached to the probe, whereas the computing is done by the APP using the processor of the smartphone.

The system is useful for scientific investigation of the brain activity. Applications also imply surveillance and analysis of a human individual's response to intake of medicine. Particular interest is monitoring conditions in connection with epilepsy, potentially including a warning mechanism to the human individual prior to an epilepsy condition. A lie-detector is another application, especially for the embodiment, where the EEG signal is combined with resistance measurements of the skin.

Other use of the apparatus is provision of stress markers. For example, an alarm signal is generated by the apparatus and either made available to the probe-bearing person and/or sent to a control station. An illustrative example is a person, such as a police officer, in stressed situation during duty, where the apparatus creates an alarm signal and transmits it to a control station, where action is taken in order to help the person in the stress situation in order to relieve the stress. For example, the action can imply activation of a microphone worn by the police officer in order to evaluate whether assistance is needed.

Stress markers can also be used to indicate to a stressed person that it is time to take a break in order to prevent long term stress.

Other use of the device is in connection with attention deficit/hyperactivity disorder (ADHD), depression, and sleep disorder.

### ASPECTS

In the following a number of interrelated aspects are listed, each of which is optionally combined with any of the other aspects mentioned herein and in the claims.

Aspect 1. A method for optimizing measurements of ElectroEncephalography, EEG, signals by EEG probes for a plurality of human individuals, the method comprising:

examining the head of each of the plurality of human individuals and providing data containing individual information related to locations of at least two head muscles for each specific of the human individuals, the at least two head muscles selected among: the anterior auricular muscle, the posterior auricular muscle, the Temporoparietalis, and the superior auricular muscle; the data comprising positions of correlation areas related to the individual information about the at least two selected muscles, wherein the correlation areas are defined as follows: an anterior correlation area A on the anterior auricular muscle, a posterior correlation area B on the posterior auricular muscle, a Temporoparietalis correlation area C on the Temporoparietalis, and a superior correlation area D on the superior auricular muscle;
providing for each of the plurality of human individuals an individually sized EEG probe with electrodes at locations on the EEG probe corresponding to the determined locations of different correlation areas for the at least two muscles;
placing the EEG probe with the electrodes against the skin of the head at the location of the different correlation areas for the at least two selected muscles;
measuring electrical signals by the electrodes;
from the measured electrical signals extracting EEG signals by correlating the signals from the at least two electrodes while reducing the electrical influence from the at least two selected muscles by selectively filtering out those parts of the signals that are uncorrelated between the electrodes.

Aspect 2. A method according to aspect 1, wherein the method comprises providing the EEG probe on only one side of the head in a region around the ear.

Aspect 3. A method according to aspect 1 or 2, wherein the method comprises providing the EEG probe as an individually sized flexible support sheet with electrodes at the locations for the different correlation areas related to the at least two selected muscles and gluing the sheet to the skin of the head such that the electrodes cover the locations for the corresponding correlation areas.

Aspect 4. A method according to any one of the aspect 1-3, wherein the method comprises providing the EEG probe with a signal processing unit attached to the EEG probe and electrically connected to the electrodes; wherein the signal processing unit comprises an A/D converter and the method comprises receiving analog raw signals from the electrodes and converting the raw signals to digital data by the A/D converter prior or after a filtering step.

Aspect 5. A method according to any one of the aspect 1-4, wherein the method comprises supplementing the EEG signals by additionally extracting ElectroCardioGraphy, ECG, signals from the electrical signals measured by the EEG probes.

Aspect 6. A method of production of plural, mutually different and individually sized EEG probes for human individuals, the method comprising:

receiving data containing individual information related to locations of at least two head muscles for each specific of the human individuals, the at least two head muscles selected among: the anterior auricular muscle, the posterior auricular muscle, the Temporoparietalis, and the superior auricular muscle; the data comprising positions of correlation areas related to the individual information about the at least two selected muscles, wherein the correlation areas are defined as follows: an anterior correlation area A on the anterior auricular muscle, a posterior correlation area B on the posterior auricular muscle, a Temporoparietalis correlation area C on the Temporoparietalis, a superior correlation area D on the superior auricular muscle;
on the basis of the received data producing an individually sized EEG probe for each of the human individuals, each EEG probe comprising electrodes at locations on the EEG probe corresponding to the individual locations of the different correlation areas for the at least two muscles according to the specific individual information for each of the human individuals.

Aspect 7. A method of production according to aspect 6, wherein the method comprises providing for each EEG probe a flexible support sheet and printing metallic ink onto the support sheet on the basis of the data for creating electrical conductors as well as electrodes at the locations corresponding to the different correlation areas for the at least two selected muscles according to the individual information.

Aspect 8. A system for measuring EEG signals by individually sized EEG probes for human individuals, the system comprising,

a digital database containing data containing individual information related to locations of at least two head muscles for each specific of the human individuals, the at least two head muscles selected among: the anterior auricular muscle, the posterior auricular muscle, the Temporoparietalis, and the superior auricular muscle; the data comprising positions of correlation areas related to the individual information about the at least two selected muscles, wherein the correlation areas are defined as follows: an anterior correlation area A on the anterior auricular muscle, a posterior correlation area B on the posterior auricular muscle, a Temporoparietalis correlation area C on the Temporoparietalis, a superior correlation area D on the superior auricular muscle;
an individually sized EEG probe for each individual, the EEG probe comprising electrodes at locations on the EEG probe corresponding to the locations of the different correlation areas for the at least two muscles according to the individual information;
a computing device for each individual, the computing device being electrically connected to the electrodes of one of the individually sized EEG probes for receiving the signals from the EEG probe and being configured for analyzing the received signals and extracting the EEG signal by correlating the signals from the at least two electrodes on the different correlation areas while reducing the electrical influence from the at least two selected muscles by selectively filtering out those parts of the signals that are uncorrelated between the electrodes.

Aspect 9. An individualized apparatus for a method according to aspect 1 or for a system according to aspect 8 configured for measuring EEG signals by an individually sized EEG probe for a human individual, the apparatus comprising,

an EEG probe individually sized for the human individual based on data containing individual information related to locations of at least two head muscles of the human individuals, specifically, the at least two head muscles selected among: the anterior auricular muscle, the posterior auricular muscle, the Temporoparietalis, and the superior auricular muscle; the data comprising positions of correlation areas related to the individual information about the at least two selected muscles, wherein the correlation areas are defined as follows: an anterior correlation area A on the anterior auricular muscle, a posterior correlation area B on the posterior auricular muscle, a Temporoparietalis correlation area C on the Temporoparietalis, and a superior correlation area D on the superior auricular muscle;
a computing device electrically connected to the electrodes of the EEG probe for receiving the signals from the EEG probe and being configured for analyzing the received signals and extracting the EEG signal by correlating the signals from the at least two electrodes on different correlation areas while reducing the electrical influence from the at least two selected muscles by selectively filtering out those parts of the signals that are uncorrelated between the electrodes.

Aspect 10. The apparatus of aspect 9, wherein the EEG probe comprises an individually sized flexible support sheet with electrodes printed by metallic ink onto the support sheet at the locations for the different correlation areas related to the at least two selected muscles and wherein the probe is configured for gluing the sheet to the skin of the head such that the electrodes cover the locations for the corresponding correlation areas.

Aspect 11. The apparatus of aspect 9, wherein a signal processing unit is attached to the EEG probe and electrically connected to the electrodes; wherein the signal processing unit comprises an A/D converter and is configured for receiving analog raw signals from the electrodes and converting the raw signals to digital data by the A/D converter prior or after a filtering step.

Aspect 12. An individualized probe for an apparatus according to aspect 9, or for a system according to aspect 8, or for a method according to aspect 1, the probe being configured for measuring EEG signals from a human individual, the EEG probe being individually sized for the human individual based on data containing individual information related to locations of at least two head muscles of the human individual, specifically, the at least two head muscles being selected among: the anterior auricular muscle, the posterior auricular muscle, the Temporoparietalis, and the superior auricular muscle; the data comprising positions of correlation areas related to the individual information about the at least two selected muscles, an anterior correlation area A on the anterior auricular muscle, a posterior correlation area B on the posterior auricular muscle, a Temporoparietalis correlation area C on the Temporoparietalis, and a superior correlation area D on the superior auricular muscle.

Aspect 13. The probe of aspect 11, wherein the EEG probe comprises an individually sized flexible support sheet with electrodes printed by metallic ink onto the support sheet at the locations for the different correlation areas related to the at least two selected muscles and wherein the probe is configured for gluing the sheet to the skin of the head such that the electrodes cover the locations for the corresponding correlation areas.

### DESCRIPTION OF THE DRAWINGS

The invention will be explained in more detail with reference to the drawing, where

FIG. 1 illustrates different prior art EEG models, where

FIG. 1a is a reproduction of the drawing from the Internet page https://emotiv.zendesk.com/hc/en-us/articles/204942089-How-come-my-headset-doesn-t-turn-on-;

FIG. 1b is a reproduction of the drawing from the Internet page http://ceegrid.com/home/concept/;

FIG. 1c is a reproduction from the Internet site http://ear-eeg.org;

FIG. 1d is a reproduction from the Internet site https://www.uneeg.com/en/products/24-7eeg/overview;

FIG. 2 shows various muscles around the ear;

FIG. 3 illustrates measure points in a system with four electrodes;

FIG. 4 illustrates measure points in a system with three electrodes;

FIG. 5 illustrates measure points in a system with two electrodes;

FIG. 6 illustrates a probe with four electrode zones, where a) illustrates the side facing the skin and b) illustrates the side remote from the skin;

FIG. 7 illustrate a probe with a signal processing unit and a wireless data transmission to a data processing unit.

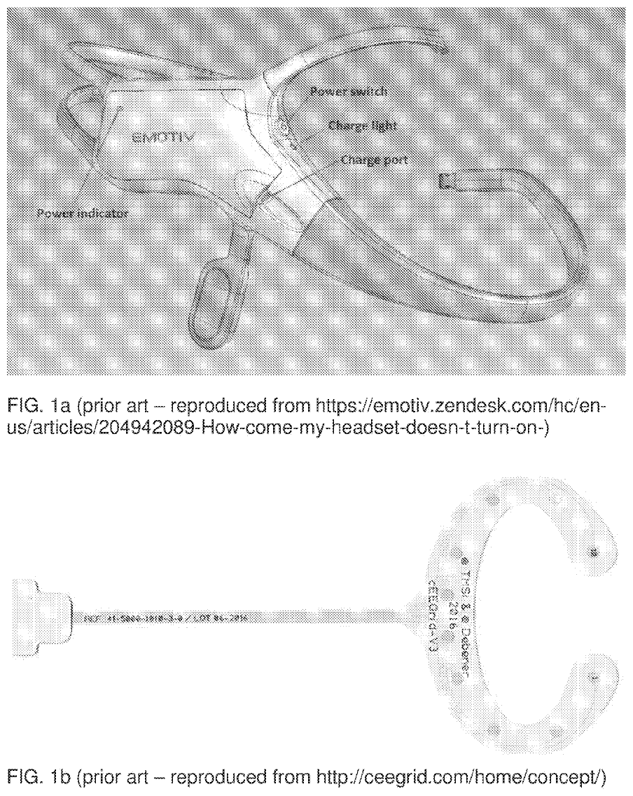

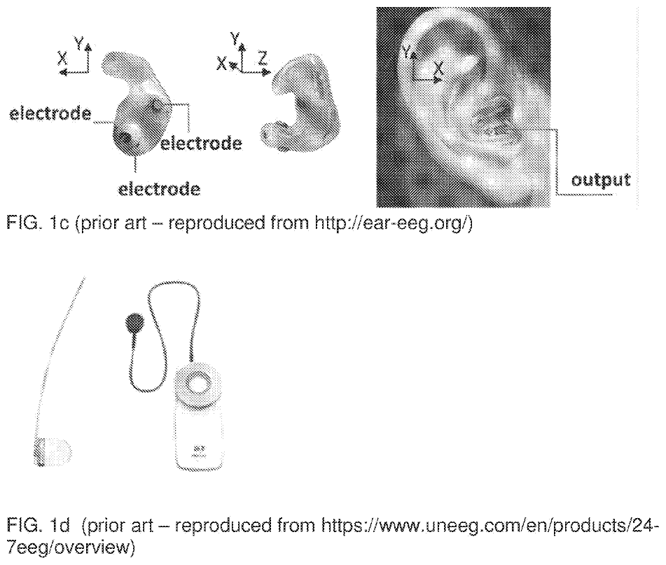

### DETAILED DESCRIPTION OF PREFERRED EMBODIMENT

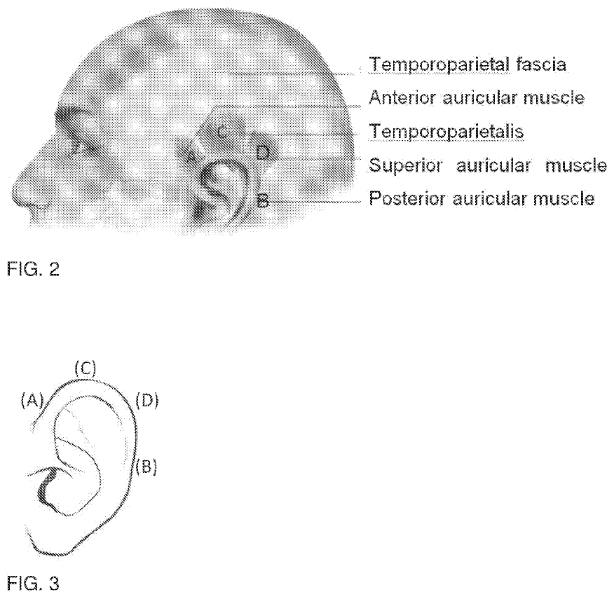

FIG. 2 illustrates various muscles around the ear. The figure is a modified reproduction from the Internet site https://plasticsurgerykey.com/scalp-and-temple/ illustrating the Temporoparietalis and the three muscles with the anterior auricular muscle in front of the ear, the posterior auricular muscle behind the ear, and the superior auricular muscle on the top.

With respect to the muscles, there are defined four correlation areas:

on the anterior auricular muscle an anterior correlation area A, for example where the pinna, soft-tissue of the ear, adheres to the anterior auricular muscle;
on the posterior auricular muscle a posterior correlation area B, for example where the posterior auricular muscle attaches to the temporal bone behind the external auditory meatus and below the zygomatic process and above the mastoid process,
on the Temporoparietalis a Temporoparietalis correlation area C, for example located where the pinna adheres on top of the Temporoparietalis muscle,
on the superior auricular muscle, a superior correlation area D, for example where the pinna adheres on top of the superior auricular muscle.

FIG. 3 illustrates positions for four electrodes coinciding with the correlation areas A, B, C, and D. By selecting correlation areas A, B, C, D for the electrodes, interference of EEG signals with electrical signals from the four muscles is minimized, as there is no signal from the same muscle in two electrodes. Due to the signals from the muscles in the four electrodes being uncorrelated, they can be easily filtered out from the raw signals from the electrodes, leaving a relatively clean correlation of EEG signals in the four electrodes.

In this embodiment, four muscle groups are probed separately as a differential gate, for example with 4 electrodes or with more than 4 electrodes, where the more than 4 electrodes are grouped into four electrode groups, each of the four electrode groups being placed in only one of the four correlation areas. For example, there are provided 4-12 electrodes, with 1-3 electrodes per muscle.

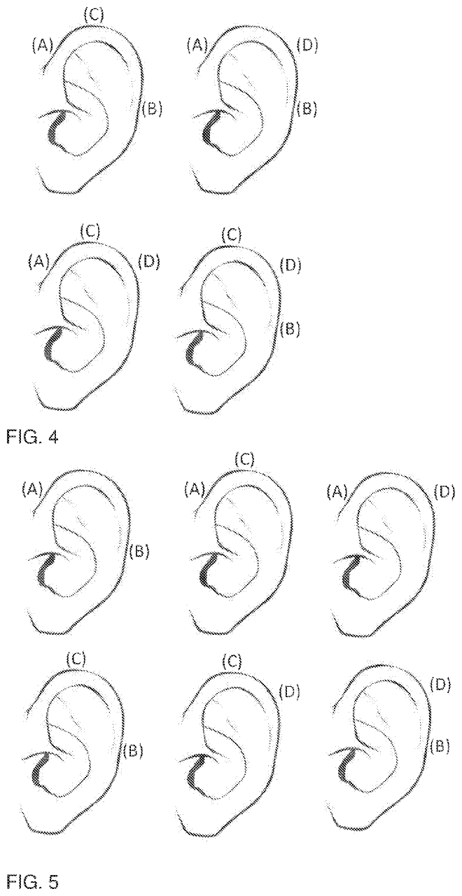

FIG. 4 illustrates a three-zone detection scheme comprising at least three electrodes or three electrode groups on three positions selected among the correlation areas A, B, C, and D. For example, three muscles are probed separately as a differential gate, for example with 3 electrode groups with 3-9 electrodes in total, with 1-3 electrodes in a group per muscle.

By reducing the number of zones as compared to the embodiment of FIG. 3, the system is simplified. However, similarly to the system in FIG. 3, two electrodes are not receiving electrical signals from the same muscle, which allows extraction of the EEG signal from the raw signals with reduced noise.

FIG. 5 illustrates a two-zone detection scheme comprising at least two separate electrodes on two selected positions among the correlation areas A, B, C, and D. Due to the decoupling of the EEG signal from the electrical signals of the muscles, it is sufficient in some cases to only use two electrodes or two groups of electrodes. For example, two muscle groups are probed separately as a differential gate, for example with 2 to 6 electrodes in total with 1-3 electrodes per muscle group.

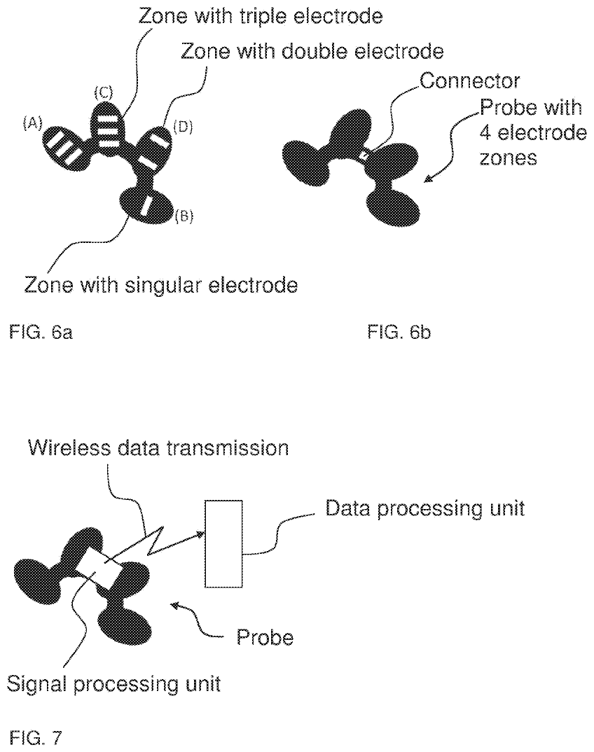

FIG. 6a illustrates a probe with four electrode zones, one zone for each of the correlation areas A, B, C, and D. For the correlation areas A and C, the corresponding zones on the probe comprise triple electrodes, for the zone at correlation area D, a double electrode is provided, and for the zone at correlation area B, a singular electrode is provided. The probe is fixated to the skin of the head around the ear, for example by using conductive adhesive on each of the electrodes.

As shown in FIG. 6b, illustrating the opposite side of the probe of FIG. 6a, a connector is provided on the probe and used for electrical connection of electronic units to the probe, for example through a wire. For example, a wire is connected to the connector and used for coupling the probe to a computing device.

Alternatively, a wearable signal processing unit is mounted and fixated onto the probe itself. Such signal processing unit is part of the aforementioned computing device and potentially wirelessly connected to a further data processing unit.

As illustrated in FIG. 7, the signal processing unit is provided in the form of a small chip attached to the connector and through the connector electronically connected to the electrodes. It is configured for receiving the raw signal from the electrodes, typically analog signals, and perform a first signal processing, for example filtering and/or analog to digital conversion. The signal processing unit is configured for transmitting the processed data to a digital data processing unit, which is also part of the computing device.

For example the data processing unit is part of a central surveillance computer, and the data are transferred wirelessly to the central surveillance computer.

Alternatively, the data processing unit is part of a small portable electronic device, such as a cell phone with a corresponding computer application, also called APP, where the digital data, as received from the signal processing unit, are further processed, for example for graphical illustration on a display with a user interface, Advantageously, the transfer of signal data in digital form from the signal processing unit is done wirelessly, for example by Bluetooth or WiFi.

Alternatively, the transfer of digital data from the signal processing unit to the data processing unit, for example the small portable device, is done by a wire. As the transfer of signal data from the signal processing unit to the data processing unit is done in digital form, there is no introduction of noise by the wire, in contrast to analog signal transmission through the wire, in which case electronic noise could be added to the analog signals from the electrodes.

1. A method of production of plural, mutually different and individually sized ElectroEncephaloGraphy, EEG, probes for human individuals, the method comprising:
receiving data containing individual information defining locations of at least two head muscles for each specific of the human individuals, the at least two head muscles selected among: the anterior auricular muscle, the posterior auricular muscle, the Temporoparietalis, and the superior auricular muscle; the data comprising positions of correlation areas related to the individual information about the at least two selected muscles, wherein the correlation areas are defined as follows: an anterior correlation area A on the anterior auricular muscle, a posterior correlation area B on the posterior auricular muscle, a Temporoparietalis correlation area C on the Temporoparietalis, a superior correlation area D on the superior auricular muscle; and
on the basis of the received data producing an individually sized EEG probe for each of the human individuals, each EEG probe comprising electrodes at locations on the EEG probe corresponding to the individual locations of the different correlation areas for the at least two muscles according to the specific individual information for each of the human individuals.

2. A method of production according to claim 1, wherein the method comprises providing for each EEG probe a flexible support sheet and printing metallic ink onto the support sheet on the basis of the data for creating electrical conductors as well as electrodes at the locations corresponding to the different correlation areas for the at least two selected muscles according to the individual information.

3. A system configured for measuring ElectroEncephaloGraphy, EEG, signals, comprising:
plural, mutually different and individually sized EEG probes for a corresponding plurality of human individuals, each EEG probe being individually sized for the corresponding human individual based on data containing individual information defining locations of at least two head muscles for each specific of the human individuals, the at least two head muscles selected among: the anterior auricular muscle, the posterior auricular muscle, the Temporoparietalis, and the superior auricular muscle;
the data comprising positions of correlation areas related to the individual information about the at least two selected muscles, wherein the correlation areas are defined as follows: an anterior correlation area A on the anterior auricular muscle, a posterior correlation area B on the posterior auricular muscle, a Temporoparietalis correlation area C on the Temporoparietalis, a superior correlation area D on the superior auricular muscle;
wherein each EEG probe comprises electrodes at locations on the EEG probe corresponding to the individual locations of the different correlation areas for the at least two muscles according to the specific individual information for each corresponding of the human individuals.

4. The system according to claim 3, further comprising a computing device electrically connected to the electrodes of the EEG probe for receiving the signals from the EEG probe and being configured for analyzing the received signals and extracting the EEG signal by correlating the signals from at least two of the electrodes on different correlation areas while reducing the electrical influence from the at least two selected muscles by selectively filtering out those parts of the signals that are uncorrelated between the electrodes.

5. The system of claim 3, wherein the EEG probe comprises an individually sized flexible support sheet with electrodes printed by metallic ink onto the support sheet at the locations for the different correlation areas related to the at least two selected muscles and wherein the probe is configured for gluing the sheet to the skin of the head such that the electrodes cover the locations for the corresponding correlation areas.

6. The system according to claim 3, wherein a signal processing unit is attached to the EEG probe and electrically connected to the electrodes; wherein the signal processing unit comprises an A/D converter and is configured for receiving analog raw signals from the electrodes and converting the raw signals to digital data by the A/D converter prior or after a filtering step.

7. The system according to claim 3, wherein the EEG probes are configured for being provided on only one side of the head in a region around the ear.

8. The system according to claim 3, wherein the system is configured for supplementing the EEG signals by additionally extracting ElectroCardioGraphy, ECG, signals from the electrical signals measured by the EEG probes.

9. A method for optimizing measurements of ElectroEncephaloGraphy, EEG, signals by the EEG probes for a plurality of human individuals, the method comprising;
providing a system according to claim 3;
providing for each of the plurality of human individuals an individually sized EEG probe with electrodes at locations on the EEG probe corresponding to the determined locations of different correlation areas for the at least two muscles;
placing the EEG probe with the electrodes against the skin of the head at the location of the different correlation areas for the at least two selected muscles;
measuring electrical signals by the electrodes;
from the measured electrical signals extracting EEG signals by correlating the signals from the at least two electrodes while reducing the electrical influence from the at least two selected muscles by selectively filtering out those parts of the signals that are uncorrelated between the electrodes.

10. The method according to claim 9, wherein the optimizing of the measurements further comprises providing the EEG probe on only one side of the head in a region around the ear.

11. The method according to claim 10, wherein the optimizing of the measurements further comprises providing the EEG probe as an individually sized flexible support sheet with electrodes at the locations for the different correlation areas related to the at least two selected muscles and gluing the sheet to the skin of the head such that the electrodes cover the locations for the corresponding correlation areas.

12. The method according to claim 9, wherein the optimizing of the measurements comprises providing the EEG probe with a signal processing unit attached to the EEG probe and electrically connected to the electrodes; wherein the signal processing unit comprises an A/D converter and the method comprises receiving analog raw signals from the electrodes and converting the raw signals to digital data by the A/D converter prior or after a filtering step.

13. The method according to claim 9, wherein the optimizing of the measurements comprises supplementing the EEG signals by additionally extracting ElectroCardioGraphy, ECG, signals from the electrical signals measured by the EEG probes.
